(19) United States
(12) Patent Application Publication
(10) Pub. No.: US 2012/0308564 A1
(43) Pub. Date: Dec. 6, 2012

(54) TREATMENT OF A METABOLIC DISORDER

(76) Inventor: Andrew Ian Bayliffe, Stevenage (GB)

(21) Appl. No.: 13/577,931

(22) PCT Filed: Feb. 7, 2011
(86) PCT No.: PCT/EP2011/051749

§ 371 Date: Aug. 9, 2012

Related U.S. Application Data
(60) Provisional application No. 61/302,637, filed on Feb. 9, 2010.

(51) Int. Cl. A61K 39/395; A61P 3/10; A61P 3/04; A61P 3/00

(52) U.S. Cl. 424/133.1; 424/158.1

(57) ABSTRACT

The present disclosure relates to the field of metabolic disorders, including type II diabetes and obesity. Specifically, the disclosure relates to methods of treating and/or preventing a metabolic disorder with an IL-18 antagonist, in particular an anti-IL-18 antigen binding protein, in particular an anti-IL-18 antibody.

### FIELD OF DISCLOSURE

The present disclosure relates to the field of metabolic disorders, including type II diabetes and obesity. Specifically, the disclosure relates to methods of treating and/or preventing a metabolic disorder with an IL-18 antagonist, in particular an anti-IL-18 antigen binding protein, in particular an anti-IL-18 antibody.

### BACKGROUND OF THE DISCLOSURE

Interleukin-18 (IL-18) is a member of the IL-1 cytokine family. IL-18 is a pleiotropic cytokine with potent effects on a diverse range of immune competent and mesenchymal cells (Nakanishe et al. (2001) Ann Rev Immunol 19: 423-427). The best characterised biological function of IL-18 is its role in host defence against microbial pathogens. IL-18 primes both innate and acquired immunity to viruses and other intracellular pathogens through activation and differentiation of Th1 and NK cells, the production of the pro-inflammatory cytokine IFN-γ (Dinarello and Boraschi (2006) Eur Cytokine Netw 17: 224-52), up-regulation of Fas and Fas ligand (FasL), and also potentiation of other proinflammatory mediators. Furthermore, IL-18 is suggested to be a potent chemotactic stimulus for human microvascular endothelial cell migration and tube formation (Park et al. (2001) J Immunol 167: 1644-1648) and, either directly or through oxidative stress pathways and matrix metalloproteins, can alter endothelial function or induce vascular smooth muscle cell migration and/or proliferation.

Pro-IL-18, the natural cellular precursor of IL-18 which is 193 amino acid residues in length, is cleaved by Caspase-1 or proteinase-3 to generate a biologically active mature 18 kDa protein which is 156 amino acid residues in length (Ghayur et al. (1997) Nature 386: 619; Gu et al (1997) Science 275: 206-208). Mature IL-18 binds to the IL-18Rα subunit resulting in the recruitment of IL-18Rβ on the cell surface. The interaction between IL-18 and the heterodimeric cell surface receptor induces signalling pathways shared with other IL-1R family members such as TLRs and IL-1 receptors (Kato et al. (2003) Nat Struct Biol 10: 966). IL-18 is expressed by macrophages, dendritic cells, osteoclasts, synovial fibroblasts, adipocytes, and epithelial cells. Whereas, IL-18R receptor is predominantly expressed on macrophages, lymphocytes, neutrophils, natural killer cells, endothelial, epithelial and smooth muscle cells (Gracie, et al. (2003) J Leukoc Biol 73: 213; Nakanishe et al. (2001) Ann Rev Immunol 19: 423-427). In vivo, the binding of IL-18 to IL-18R complex is regulated by IL-18 binding protein (IL-18BP). The IL-18BP is constitutively expressed and acts as a natural inhibitor of IL-18 functions.

The postulated role of IL-18 in the development of autoimmune diseases is supported by the following findings. IL-18 is elevated in various target tissues associated with a range of autoimmune diseases, most notably in Adult Onset Stills Disease (AOSD) (plasma and liver) (Kawashima et al. (2001) Arthritis Rheum 44: 550-560), Systemic Lupus Erythematosus (SLE) (plasma and various tissues), Rheumatoid Arthritis (RA) (plasma and synovium) (Tanaka et al. (2004) Life Sciences 74: 1671-1674), Crohn's disease (plasma and gut epithelium) (Pizarro et al. (1999) J Immunol 162: 6829-6835) and Psoriasis (plasma and skin) (Ohta et al. (2001) 293: 334-342).

More recently, IL-18 levels in the periphery have also been found to correlate with body weight and insulin resistance, with IL-18 having the potential to predict progression to type 2 diabetes mellitus (T2DM) (Murdolo et al. (2008) Am J Physiol Endocrinol Metab 295: E1095-E1105; Fischera et al. (2005) Clinical Immunology 117: 152-160). In addition, circulating levels of IL-18 also appear to be causally implicated in a number of co-morbidities of obesity and T2DM. In particular, it has been shown that plasma levels of IL-18 correlate with intimal-medial thickening and predict future cardiovascular events in T2DM patients [Yamagami et al. (2005) Arterioscler Thromb Vasc Biol. 25: 1458-1462].

T2DM is characterised by peripheral insulin resistance e.g. cells fail to respond to insulin properly, increased hepatic glucose production and impaired insulin secretion. The prevalence of T2DM has increased considerably in recent years due to alterations in dietary patterns (higher levels of obesity) as well as changes in lifestyles (more sedentary), reaching epidemic proportions. The first line of treatment for T2DM is diet, weight control and increased physical activity. However, if these approaches are not successful in reducing blood glucose levels, patients may be prescribed glucose lowering medication, such as metformin, or may need insulin injections. A number of other treatments are used to control T2DM and these include PPAR gamma agonists such as rosiglitazone, GLP1 receptor agonists (e.g. exenatide (Byetta™), liraglutide (Victoza™)) and PYY receptor agonists.

There are no anti-obesity agents, alone or in combination, currently on the market or in development, that deliver more than 10% weight loss as compared to placebo. In addition, many marketed drugs suffer from severe tolerability issues such as nausea and vomiting. The number of small molecules and biologicals in development is increasing. Of particular note, Qnexa™, a combination of phentermine and topiramate, demonstrated 11% weight loss vs. 1.6% (4 lbs) in a placebo controlled PhIII trial although still with significant side effects. In addition, peptide combinations (Glucagon/GLP-1 co-agonist, and GLP-1/oxyntomodulin co-agonist: Merck) are currently in pre-clinical investigation and both demonstrate significant weight loss though may still be associated with side effects.

In contrast to T2DM, type 1 diabetes mellitus (T1DM) affects 5-10% of diabetes sufferers and results from the body's failure to produce insulin as a result of loss of β-cells in the islet of Langerhans in the pancreas. Sufferers of T1DM require treatment in the form of insulin injections.

Investigations into IL-18 antagonists in the field of diabetes have focussed on the type 1 (autoimmune) diabetes. For example, the antagonistic IL-18 binding protein (IL-18BP) has been shown to attenuate the progression of diabetes in the non-obese mouse (NOD) model of T1DM when dosed prophylactically to young mice. The authors postulate a possible protective effect of IL-18BP on β-cell apoptosis (Zacconea and Phillips (2005) Clinical Immunology 115: 74-79). Moreover, IL-18BP has been shown to protect β-cells from apoptosis in ex vivo β-cell destruction assays (Lewis and Dinarello (2006) PNAS 103: 16852-16857).

Whilst IL-18 in serum and peripheral tissues has been shown to be significantly elevated and correlated with onset of insulin resistance (IR) in obese mice fed a high-fat diet (T2DM model), there is no evidence of any investigations into the impact of IL-18 antagonists in these models in the published literature.

IL-18 knock-out mice have been studied. IL-18 knock-out mice are hyperphagic and become obese and insulin resistant. A recent metabolic analysis of IL-18 knock-out mice has shown that reconstitution of IL-18 knock-out mice with intracranial doses of murine IL-18 restores normal feeding behaviour and subsequently results in weight loss and normal glycaemic control, whereas intravenous (IV)/intraperitoneal (IP) murine IL-18 had no discernable effect. In light of this data, a central role for IL-18 in regulating feeding behaviour, implicating the hypothalamus as a target organ, either directly or indirectly, for the action of IL-18 in promoting a satiety response, has been suggested (Zorilla et al. (2007) PNAS 104: 11097-11102; Natea and Joosten (2006) Nat Med 12: 650-656). It has been proposed that obesity and insulin resistance are secondary effects arising from induction of hyperphagia in these mice.

The findings in IL-18 knock-out mice sit paradoxically with data linking elevated IL-18 levels with increased severity of human metabolic disease, and may suggest that, rather than being causal in metabolic diseases, elevated expression of IL-18 in humans may be a compensatory response.

Accordingly, the precise role of IL-18, particularly with respect to metabolic disease, is far from clear and cause-and-effect has not been established for IL-18 in models of metabolic disease or in the clinic.

### SUMMARY OF THE DISCLOSURE

There is a need for safe and improved treatments and preventative measures for metabolic diseases such as T2DM, obesity-related T2DM, and obesity, given the prevalence of these disorders. There are few effective pharmacological interventions for T2DM and most are associated with poor efficacy and significant tolerability issues. In addition, weight is typically regained once therapy ceases and often existing treatments fail to prevent a decrease in β-cell function and eventual β-cell destruction with a switch from T2DM to T1DM.

An aim of the present disclosure is to provide a new and improved treatment for metabolic diseases, particularly a new treatment for T2DM that reduces body weight, improves glycaemic control, increases insulin sensitivity without loss of β-cell function and, as a result, slows disease progression. A further aim of the present disclosure is to improve other T2DM co-morbidities such as cardiovascular health.

The present disclosure provides, in a first aspect, an IL18 antagonist for use in treating or preventing a metabolic disorder in a patient and/or improving glycaemic control in a patient.

The present disclosure provides, in a second aspect, a method of treating a patient afflicted with a metabolic disorder by administering a therapeutically effective amount of an IL-18 antagonist to said patient.

The present disclosure provides, in a third aspect, a method of preventing a metabolic disorder in a patient susceptible to such a metabolic disorder by administering a prophylactically effective amount of an IL-18 antagonist to said patient.

The present disclosure provides, in a fourth aspect, a method of improving glycaemic control in a patient by administering a therapeutically effective amount of an IL-18 antagonist to said patient.

### BRIEF DESCRIPTION OF THE FIGURES

FIGS. 1-10 show the intensity values (on the log scale) for ex-vivo whole blood samples from 10 healthy donors treated with either: A) Synagis™ (anti-RSV) IgG 1 ug/ml, B) IL-18 50 ng/ml, C) IL-18 50 ng/ml+H1L2 1 ug/ml or D) H1L2 1 ug/ml, for IL-6, JAK2, SOCS3, STAT3, MCP-1 (CCL2), MCP-4 (CCL13), IRS2, PPAR gamma, LEP and LEPR. Data points are shaded by donor.

### DETAILED DESCRIPTION OF THE DISCLOSURE

The disclosure hypothesizes that insulin resistance is an inflammatory disorder and that inflammatory mediators synthesised from cells of the immune system, as well as by adipose tissue, are involved in the regulation of insulin. The postulates that over-nutrition and obesity lead to a low chronic inflammatory state which results in an increase in inflammatory marker expression which in turn has the consequence of increasing infiltration of macrophages into fat tissue. These macrophages secrete proinflammatory cytokines which impair insulin signalling in adipocytes and subsequently increase lipolysis and release of fatty acids into the circulation. Fatty acids render the liver and skeletal muscle insulin resistant and contribute to a pre-diabetic state which leads to the development of diabetes.

Surprisingly, the Applicants have found that IL-18 induces expression of Interleukin-6 (IL-6) and its key signalling molecules (e.g. Janus kinase 2-JAK2) in human blood and that these effects can be neutralised by an IL18 antagonist of the disclosure i.e. H1L2 (SEQ ID NO:7 and SEQ ID NO:11).

IL-6 is a key mediator of chronic inflammation and has been implicated in obesity, insulin resistance and T2DM. Adipose tissue can contribute up to 35% of circulating IL-6 levels. The systemic effects of chronic low level IL-6 expression can inhibit insulin function through signal transducer and activator of transcription 3 (STAT3) and suppressor of cytokine signalling 3 (SOCS3) expression. JAK2 and STAT3 increase expression of SOCS3, which can prevent insulin receptor (IR) activation of insulin receptor substrate (IRS; also decreased by IL-18) reducing uptake of circulating glucose by muscles and adipose, and reducing glycogen availability (Kim et al. (2009) Vitamins and Hormones 80: 613-633).

Accordingly, the Applicants' observations suggest, for the first time, that an IL-18 antagonist may represent a useful treatment for disorders in which IL-6 levels are or IL-6 expression is elevated compared to normal levels or expression in healthy individuals e.g. metabolic disorders such as T2DM, obese T2DM and obesity.

In addition the Applicants have shown that an IL-18 antagonist down-regulates other important inflammatory mediators, including monocyte chemoattractant proteins (MCP-1 and MCP-4). These chemotactic proteins are increased in genetically obese mice and healthy mice in which obesity has been induced through a high fat diet. It has been proposed that MCP-1 and MCP-4 link obesity and insulin resistance by the induction of a low-grade inflammatory response (macrophage infiltration) in adipose tissue in obese subjects.

Further, the Applicants have shown that IL-18 decreases expression of IRS2 in human blood and that these effects can be neutralised by an IL18 antagonist of the disclosure i.e. H1L2 (SEQ ID NO:7 and SEQ ID NO:11).

Insulin signalling is coordinated with counter-regulatory signalling through tyrosine phosphorylation of the insulin receptor substrates IRS1-4 with IRS-2 being especially important in nutrient homeostasis. IRS-2 is the major effector of the metabolic and growth-promoting effects of insulin and promotes pancreatic beta cell function and survival and central nutrient sensing (Dong et al., (2006) J. Clin. Invest., 116(1): 101-104). The conditional knockout of IRS-2 in mice increases appetite, lean and fat body mass and linear growth with eventual progression to diabetes. In addition, IRS-2 knockdown results in fasting hyperglycaemia, fasting hyperinsulinaemia, insulin resistance, glucose intolerance, dyslipidaemia and other characteristics consistent with metabolic syndrome (Taniguchi et al., (2005) J. Clin. Invest. 115(3): 718-727). These data suggest that down-regulation of IRS-2 (by increased IL-18 as supported by the ex-vivo blood assay) may play an important role play in the onset of obesity in man and the subsequent progression to T2DM. Blockade of IL-18's effects with an anti-IL-18 antagonist may reverse IRS-2 dysfunction.

The Applicants have also shown that an anti-IL-18 antagonist up-regulates PPAR gamma, an orphan receptor highly expressed in adipose tissue. PPAR gamma agonists such as rosiglitazone, have been used in the treatment of T2DM as they have been shown to improve insulin sensitivity.

The Applicants have also shown that an anti-IL-18 antagonist up-regulates leptin and the leptin receptor. Much evidence links low levels of leptin, an appetite suppressing hormone, and the leptin receptor to obesity. It is suggested that leptin resistance may result in obesity and leptin has been used in the treatment of obesity. Up-regulation of leptin and its receptor support a role for an anti-IL-18 antagonist in appetite suppression and subsequently a reduction in body weight in obese subjects and T2DM patients.

We have also surprisingly shown that there is a link between IL-18 levels in human patients and elevated plasma glucose levels. Accordingly, an IL-18 antagonist may represent a useful treatment for disorders in which plasma glucose is elevated compared to normal levels in healthy individuals e.g. metabolic disorders such as T2DM, obese T2DM and obesity.

Indeed we have shown in a separate study that H1L2 modulates various metabolic parameters, including glucose and insulin in obese, but otherwise healthy, humans.

In light of our findings we predict that by blocking the function of peripheral IL-18 in obese and/or diabetic human patients and subsequently preventing low-grade inflammation, serum and plasma glucose levels will be reduced and insulin resistance will be attenuated. By blocking IL-18 action in the periphery, body weight and adiposity should be impacted. This may impact body weight and improve glycaemic control without adversely affecting β-cell function. Indeed, an IL-18 antagonist may have direct protective effects of β-cell function by reducing apoptosis. In addition, a favourable impact on cardiovascular co-morbidities is envisioned.

“Glycaemic control” as used throughout the specification refers to the typical levels of blood glucose in a patient with diabetes mellitus, compared with the normal levels of blood glucose seen in a healthy individual, and said patient's ability to control these levels. Poor glycaemic control refers to persistently elevated blood glucose above the normal levels and perfect glycaemic control refers to blood glucose levels always within the normal range.

An “IL-18 antagonist” as used herein is an agent that inhibits or antagonises, to some extent, a biological activity of IL-18. IL-18 antagonists include agents which bind to IL-18, such as the endogenous IL-18 binding proteins (IL-18BP) when isolated from the body or recombinantly produced (e.g. Tadekinig-α®), as well as IL-18BP-Fc fusion proteins, or antagonists which bind to a receptor for IL-18 and thereby prevent IL-18 from exerting its biological activity. Specifically contemplated IL-18 antagonists are anti-IL-18 antigen binding proteins, e.g. antibodies, that are immunospecific for IL-18, and that antagonise an activity of IL-18. Non-limiting examples of IL-18 antagonists include H1 and H2 described in European patent EP0712931, H18-108 (Hamasaki et al., 2005), and the antibodies described in WO01/58956, WO2005/047307 and WO2007/137984, all of which are herein incorporated by reference in their entirety. In an embodiment, the IL-18 antagonist is a protein, such as an isolated or recombinantly produced IL-18BP, or an IL-18 antigen binding protein. In a further embodiment, the IL-18 antagonist is an antigen binding protein. In an embodiment, the IL-18 antagonist is not a new chemical entity (NCE). In a particular embodiment, the IL-18 antigen binding protein is the antibody H1L2 as disclosed herein (SEQ ID NO:7 and SEQ ID NO:11) or a variant thereof.

The term “anti-IL-18”, as it refers to antigen binding proteins of the disclosure, means that such antibodies are capable of neutralising a biological activity of human IL-18. It does not exclude, however, that such antibodies may also in addition neutralise the biological activity of non-human primate (e.g. rhesus and/or cynomolgus) IL-18 and/or forms of IL-18 present in other species.

The term “antibody” is used herein in the broadest sense to refer to molecules with an immunoglobulin-like domain and includes monoclonal, recombinant, polyclonal, chimeric, humanised, multispecific e.g. bispecific and heteroconjugate antibodies; a single variable domain, a domain antibody, antigen binding fragments, immunologically effective fragments, single chain Fv, diabodies, Tandabs™, etc. (for a summary of alternative “antibody” formats see Holliger and Hudson, Nature Biotechnology, 2005, Vol 23, No. 9, 1126-1136).

The phrase “single variable domain” refers to an antigen binding protein variable domain (for example, VH, VHH, VL) that specifically binds an antigen or epitope independently of a different variable region or domain.

A “domain antibody” or “dAb™” may be considered the same as a “single variable domain” which is capable of binding to an antigen. A single variable domain may be a human antibody variable domain, but also includes single antibody variable domains from other species such as rodent (for example, as disclosed in WO 00/29004), nurse shark and Camelid VHH dAb™s. Camelid VHH are immunoglobulin single variable domain polypeptides that are derived from species including camel, llama, alpaca, dromedary, and guanaco, which produce heavy chain antibodies naturally devoid of light chains. Such VHH domains may be humanised according to standard techniques available in the art, and such domains are considered to be “domain antibodies”. As used herein VH includes camelid VHH domains.

As used herein the term “domain” refers to a folded protein structure which has tertiary structure independent of the rest of the protein. Generally, domains are responsible for discrete functional properties of proteins, and in many cases may be added, removed or transferred to other proteins without loss of function of the remainder of the protein and/or of the domain. A “single variable domain” is a folded polypeptide domain comprising sequences characteristic of antibody variable domains. It therefore includes complete antibody variable domains and modified variable domains, for example, in which one or more loops have been replaced by sequences which are not characteristic of antibody variable domains, or antibody variable domains which have been truncated or comprise N- or C-terminal extensions, as well as folded fragments of variable domains which retain at least the binding activity and specificity of the full-length domain. A domain can bind an antigen or epitope independently of a different variable region or domain.

An antigen binding fragment may be provided by means of arrangement of one or more CDRs on non-antibody protein scaffolds such as a domain. The domain may be a domain antibody or may be a domain which is a derivative of a scaffold selected from the group consisting of CTLA-4, lipocalin, SpA, an Affibody, an avimer, GroEI, transferrin, GroES and fibronectin/adnectin, which has been subjected to protein engineering in order to obtain binding to an antigen, such as IL-18, other than the natural ligand.

An antigen binding fragment or an immunologically effective fragment may comprise partial heavy or light chain variable sequences. Fragments are at least 5, 6, 7, 8, 9 or 10 amino acids in length. Alternatively the fragments are at least 15, at least 20, at least 50, at least 75, or at least 100 amino acids in length.

The term “specifically binds” as used in relation to antigen binding proteins means that the antigen binding protein binds to IL-18 with no or insignificant binding to other (for example, unrelated) proteins.

The term “immunospecific” as used in relation to an antibody means an antibody that binds its target protein (e.g. human IL-18) with no or insignificant binding to other proteins. The term, however, does not exclude the fact that an antibody to a target protein in a given species (e.g. human) may also be cross-reactive with other forms of the target protein in other species (e.g. a non-human primate).

The equilibrium dissociation constant (KD) of the antigen binding protein-IL-18 interaction may be 1 mM or less, 100 nM or less, 10 nM or less, 2 nM or less or 1 nM or less. Alternatively the KD may be between 5 and 10 nM; or between 1 and 2 nM. The KD may be between 1 pM and 500 pM; or between 500 pM and 1 nM. The binding affinity may be measured by BIAcore™, for example by antigen capture with IL-18 coupled onto a CM5 chip by primary amine coupling and antibody capture onto this surface. Alternatively, the binding affinity can be measured by FORTEbio, for example by antigen capture with IL-18 coupled onto a CM5 needle by primary amine coupling and antibody capture onto this surface. H1L2 (SEQ ID NO:7 and SEQ ID NO:11) binds human IL-18 with high affinity (KD=30.3 pM). In a particular embodiment, the equilibrium dissociation constant with respect to an anti-IL-18 antigen binding protein binding of the disclosure and human IL-18 is about 30 pM, or less than 30 pM, when measured at 25° C.

The term “neutralises” as used in the present specification means that the biological activity of IL-18 is reduced in the presence of an antigen binding protein as described herein in comparison to the activity of IL-18 in the absence of the antigen binding protein, in vitro or in vivo. Neutralisation may be due to one or more of blocking IL-18 binding to the IL-18 receptor, clearing IL-18 from the circulation, down regulating IL-18 or the IL-18 receptor, or affecting effector functionality.

IL-18 activity can be indirectly measured using an interferon-γ (IFN-γ) assay in ex-vivo stimulated whole blood. Briefly, 30 mls blood is collected into standard citrate or heparin anticoagulant and the following protocol is used. Aliquot ˜3 μl of treatment directly into the wells of a 6-well plate (treatments should include 50 ng/ml IL18 and an appropriate control). Add 3 mls of whole blood to each well and mix by shaking gently. Incubate plate at 37° C. in a CO₂ incubator for 4 hours mixing gently on a shaker every 15 minutes. At the end of the incubation transfer 2.5 mls blood to a PAX tube, invert PAX tube 8-10 times and store upright at room temperature for 2 hours. Transfer PAX tubes to −20° C. for medium term storage. IFN-γ levels can be measured using either TaqMan or ELISA/MSD following manufacturers' guidelines.

A “chimeric antibody” refers to a type of engineered antibody which contains a naturally-occurring variable region (light chain and heavy chains) derived from a donor antibody in association with light and heavy chain constant regions derived from an acceptor antibody.

A “humanised antibody” refers to a type of engineered antibody having its CDRs derived from a non-human donor immunoglobulin, the remaining immunoglobulin-derived parts of the molecule being derived from one or more human immunoglobulin(s). In addition, framework support residues may be altered to preserve binding affinity (see, e.g., Queen et al. Proc. Natl Acad Sci USA, 86:10029-10032 (1989), Hodgson et al. Bio/Technology, 9:421 (1991)). A suitable human acceptor antibody may be one selected from a conventional database, e.g., the KABAT® database, Los Alamos database, and Swiss Protein database, by homology to the nucleotide and amino acid sequences of the donor antibody. A human antibody characterized by a homology to the framework regions of the donor antibody (on an amino acid basis) may be suitable to provide a heavy chain constant region and/or a heavy chain variable framework region for insertion of the donor CDRs. A suitable acceptor antibody capable of donating light chain constant or variable framework regions may be selected in a similar manner. It should be noted that the acceptor antibody heavy and light chains are not required to originate from the same acceptor antibody. The prior art describes several ways of producing such humanised antibodies—see for example EP-A-0239400 and EP-A-054951. In an embodiment, an antibody of the disclosure is a humanised antibody.

The term “human antibody” refers to an antibody derived from human immunoglobulin gene sequences. These fully human antibodies provide an alternative to re-engineered, or de-immunized, rodent monoclonal antibodies (e.g. humanised antibodies) as a source of low immunogenicity therapeutic antibodies and they are normally generated using either phage display or transgenic mouse platforms In an embodiment, an antibody of the disclosure is a human antibody.

The terms “VH” and “VL” are used herein to refer to the heavy chain variable region and light chain variable region respectively of an antigen binding protein.

“CDRs” are defined as the complementarity determining region amino acid sequences of an antigen binding protein. These are the hypervariable regions of immunoglobulin heavy and light chains. There are three heavy chain CDRs and three light chain CDRs (or CDR regions) in the variable portion of an immunoglobulin. Thus, “CDRs” as used herein refers to all three heavy chain CDRs, all three light chain CDRs, all heavy and light chain CDRs, or at least two CDRs.

Throughout this specification, amino acid residues in variable domain sequences and full length antibody sequences are numbered according to the Kabat numbering convention. The terms “CDR”, “CDRL1”, “CDRL2”, “CDRL3”, “CDRH1”, “CDRH2”, “CDRH3” in relation to specific sequences disclosed herein also follow the Kabat numbering convention. For further information, see Kabat et al., Sequences of Proteins of Immunological Interest, 4th Ed., U.S. Department of Health and Human Services, National Institutes of Health (1987).

However, although we use the Kabat numbering convention for amino acid residues in variable domain sequences and full length antibody sequences throughout this specification, it will be apparent to those skilled in the art that there are alternative numbering conventions for amino acid residues in variable domain sequences and full length antibody sequences. There are also alternative numbering conventions for CDR sequences, for example those set out in Chothia et al. (1989) Nature 342: 877-883. The structure and protein folding of the antibody may mean that other residues are considered part of the CDR sequence and would be understood to be so by a skilled person.

Other numbering conventions for CDR sequences available to a skilled person include “AbM” (University of Bath) and “contact” (University College London) methods. The minimum overlapping region using at least two of the Kabat, Chothia, AbM and contact methods can be determined to provide the “minimum binding unit”. The minimum binding unit may be a sub-portion of a CDR.

Table 1 below represents one definition using each numbering convention for each CDR or binding unit. The Kabat numbering scheme is used in Table 1 to number the variable domain amino acid sequence. It should be noted that some of the CDR definitions may vary depending on the individual publication used.

TABLE 1

| | | | | | Minimum |
|---|---|---|---|---|---|
| | Kabat | Chothia | AbM | Contact | binding |
| | CDR | CDR | CDR | CDR | unit |
| H1 | 31-35/ | 26-32/ | 26-35/ | 30-35/ | 31-32 |
| | 35A/35B | 33/34 | 35A/35B | 35A/35B | |
| H2 | 50-65 | 52-56 | 50-58 | 47-58 | 52-56 |
| H3 | 95-102 | 95-102 | 95-102 | 93-101 | 95-101 |
| L1 | 24-34 | 24-34 | 24-34 | 30-36 | 30-34 |
| L2 | 50-56 | 50-56 | 50-56 | 46-55 | 50-55 |
| L3 | 89-97 | 89-97 | 89-97 | 89-96 | 89-96 |

In an embodiment, an antigen binding protein of the disclosure comprises the CDRs contained within SEQ ID NO:7 and/or SEQ ID NO: 11. In an embodiment, an antigen binding protein of the disclosure comprises any one of more of the following CDRs or a variant thereof: CDRH1 (SEQ ID NO:1), CDRH2 (SEQ ID NO:2), CDRH3 (SEQ ID NO:3), CDRL1 (SEQ ID NO:4), CDRL2 (SEQ ID NO:5), CDRL3 (SEQ ID NO:6). In an embodiment, an antigen binding protein of the disclosure comprises CDRH1 (SEQ ID NO:1), CDRH2 (SEQ ID NO:2), CDRH3 (SEQ ID NO:3), CDRL1 (SEQ ID NO:4), CDRL2 (SEQ ID NO:5), and CDRL3 (SEQ ID NO:6).

One or more of the CDRs or variant CDRs described herein may be present in the context of a human framework, for example as a humanised or chimeric variable domain.

A “CDR variant” includes an amino acid sequence modified by at least one amino acid, wherein said modification can be chemical or a partial alteration of the amino acid sequence (for example by no more than 10 amino acids), which modification permits the variant to retain the biological characteristics of the unmodified sequence. For example, the variant is a functional variant which binds to and neutralises IL-18. A partial alteration of the CDR amino acid sequence may be by deletion or substitution of one to several amino acids, or by addition or insertion of one to several amino acids, or by a combination thereof (for example by no more than 10 amino acids). The CDR variant may contain 1, 2, 3, 4, 5 or 6 amino acid substitutions, additions or deletions, in any combination, in the amino acid sequence. The CDR variant or binding unit variant may contain 1, 2 or 3 amino acid substitutions, insertions or deletions, in any combination, in the amino acid sequence. The substitutions in amino acid residues may be conservative substitutions, for example, substituting one hydrophobic amino acid for an alternative hydrophobic amino acid. For example leucine may be substituted with valine, or isoleucine.

In an embodiment, the anti-IL-18 antibody is selected from the group consisting of: H1L2 (SEQ ID NO:7 and SEQ ID NO:11), ABT-325 (Abbott), and 125-2H (R&D Systems).

For nucleotide and amino acid sequences, the term “identical” or “sequence identity” indicates the degree of identity between two nucleic acid or two amino acid sequences when optimally aligned and compared with appropriate insertions or deletions.

The percent identity between two sequences is a function of the number of identical positions shared by the sequences (i.e., % identity=number of identical positions/total number of positions multiplied by 100), taking into account the number of gaps, and the length of each gap, which need to be introduced for optimal alignment of the two sequences. The comparison of sequences and determination of percent identity between two sequences can be accomplished using a mathematical algorithm, as described below.

The percent identity between two nucleotide sequences can be determined using the GAP program in the GCG software package, using a NWSgapdna.CMP matrix and a gap weight of 40, 50, 60, 70, or 80 and a length weight of 1, 2, 3, 4, 5, or 6. The percent identity between two nucleotide or amino acid sequences can also be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. 48:444-453 (1970)) algorithm which has been incorporated into the GAP program in the GCG software package, using either a Blossum 62 matrix or a PAM250 matrix, and a gap weight of 16, 14, 12, 10, 8, 6, or 4 and a length weight of 1, 2, 3, 4, 5, or 6.

A polypeptide sequence may be identical to a polypeptide reference sequence as described herein (see for example SEQ ID NO: 7), that is be 100% identical, or it may include up to a certain integer number of amino acid alterations as compared to the reference sequence such that the % identity is less than 100%, such as at least 50, 60, 70, 75, 80, 85, 90, 95, 96, 97, 98, or 99% identical. Such alterations are selected from the group consisting of at least one amino acid deletion, substitution, including conservative and non-conservative substitution, or insertion, and wherein said alterations may occur at the amino- or carboxy-terminal positions of the reference polypeptide sequence or anywhere between those terminal positions, interspersed either individually among the amino acids in the reference sequence or in one or more contiguous groups within the reference sequence. The number of amino acid alterations for a given % identity is determined by multiplying the total number of amino acids in the polypeptide sequence encoded by the polypeptide reference sequence as described herein (see for example SEQ ID NO:7) by the numerical percent of the respective percent identity (divided by 100) and then subtracting that product from said total number of amino acids in the polypeptide reference sequence as described herein (see for example SEQ ID NO: 7), or:

nₐ≦xₐ−(xₐ·y),

wherein nₐ is the number of amino acid alterations, xₐ is the total number of amino acids in the reference polypeptide sequence as described herein (see for example SEQ ID NO:7), and y is, 0.50 for 50%, 0.60 for 60%, 0.70 for 70%, 0.75 for 75%, 0.80 for 80%, 0.85 for 85%, 0.90 for 90%, 0.95 for 95%, 0.98 for 98%, 0.99 for 99%, or 1.00 for 100%, · is the symbol for the multiplication operator, and wherein any non-integer product of xₐ and y is rounded down to the nearest integer prior to subtracting it from xₐ.

The % identity may be determined across the length of the sequence.

An antibody heavy chain of the disclosure may have 75% or greater, 80% or greater, 85% or greater, 90% or greater, 95% or greater, 96% or greater, 97% or greater, 98% or greater, 99% or greater or 100% identity to SEQ ID NO: 7 (heavy chain H1), SEQ ID NO:8 (heavy chain H2), or SEQ ID NO:9 (heavy chain H3). In a particular embodiment, the antibody heavy chain of the disclosure has 75% or greater, 80% or greater, 85% or greater, 90% or greater, 95% or greater, 96% or greater, 97% or greater, 98% or greater, 99% or greater or 100% identity to SEQ ID NO: 7 (heavy chain 1).

An antibody light chain of the disclosure may have 75% or greater, 80% or greater, 85% or greater, 90% or greater, 95% or greater, 96% or greater, 97% or greater, 98% or greater, 99% or greater, or 100% identity to SEQ ID NO:10 (light chain L1), SEQ ID NO:11 (light chain L2), or SEQ ID NO:12 (light chain L3). In a particular embodiment, the antibody light chain of the disclosure has 75% or greater, 80% or greater, 85% or greater, 90% or greater, 95% or greater, 96% or greater, 97% or greater, 98% or greater, 99% or greater, or 100% identity to SEQ ID NO:11 (light chain L2).

An antibody heavy chain of the disclosure may be a variant of SEQ ID NO:7, SEQ ID NO:8, or SEQ ID NO:9, which contains 30, 25, 20, 15, 10, 9, 8, 7, 6, 5, 4, 3, 2 or 1 amino acid substitutions, insertions or deletions. In an embodiment, the antibody heavy chain is a variant of SEQ ID NO:7. An antibody light chain of the disclosure may be a variant of SEQ ID NO:10, SEQ ID NO:11, or SEQ ID NO:12 which contains 30, 25, 20, 15, 10, 9, 8, 7, 6, 5, 4, 3, 2 or 1 amino acid substitutions, insertions or deletions. In an embodiment, the antibody light chain is a variant of SEQ ID NO:11.

The terms “peptide”, “polypeptide” and “protein” each refers to a molecule comprising two or more amino acid residues. A peptide may be monomeric or polymeric.

It is well recognised in the art that certain amino acid substitutions are regarded as being “conservative”. Amino acids are divided into groups based on common side-chain properties and substitutions within groups that maintain all or substantially all of the binding affinity of the antigen binding protein are regarded as conservative substitutions, see Table 2 below:

TABLE 2

| Side chain | Members |
|---|---|
| Hydrophobic | met, ala, val, leu, ile |
| Neutral hydrophilic | cys, ser, thr |
| Acidic | asp, glu |
| Basic | asn, gln, his, lys, arg |
| Residues that influence chain orientation | gly, pro |
| Aromatic | trp, tyr, phe |

In an embodiment, an antigen binding protein of the disclosure specifically binds to and neutralises IL-18 and competes for binding to IL-18 with a reference antibody comprising a heavy chain sequence of SEQ ID NO: 7 and a light chain sequence of SEQ ID NO: 11 (H1L2).

Competition between the antigen binding protein and the reference antibody may be determined by competition ELISA. A competing antigen binding protein may bind to the same epitope, an overlapping epitope, or an epitope in close proximity of the epitope to which the reference antibody binds.

The antigen binding protein may be derived from rat, mouse, primate (e.g. cynomolgus, Old World monkey or Great Ape) or human. The antigen binding protein may be a humanised or chimeric antibody. The antigen binding protein may be a human antibody.

The antigen binding protein may comprise a constant region, which may be of any isotype or subclass. The constant region may be of the IgG isotype, for example IgG1, IgG2, IgG3, IgG4 or variants thereof. In an embodiment, the antigen binding protein constant region is IgG1.

The antigen binding protein comprising a constant domain region may have reduced ADCC and/or complement activation or effector functionality. The constant domain may comprise a naturally disabled constant region of IgG2 or IgG4 isotype or a mutated IgG1 constant domain. Examples of suitable modifications are described in EP0307434. One example comprises the substitutions of alanine residues at positions 235 and 237 (EU index numbering).

The antigen binding protein may comprise one or more modifications selected from a mutated constant domain such that the antibody has enhanced effector functions/ADCC and/or complement activation. Examples of suitable modifications are described in Shields et al. J. Biol. Chem (2001) 276: 6591-6604, Lazar et al. PNAS (2006) 103: 4005-4010 and U.S. Pat. No. 6,737,056, WO2004063351 and WO2004029207.

The antigen binding protein may comprise a constant domain with an altered glycosylation profile such that the antigen binding protein has enhanced effector functions/ADCC and/or complement activation. Examples of suitable methodologies to produce an antigen binding protein with an altered glycosylation profile are described in WO2003/011878, WO2006/014679 and EP1229125.

Purified preparations of an Il-18 antagonist, e.g. antigen binding protein, as described herein may be incorporated into pharmaceutical compositions for use in the treatment of the human diseases, disorders and conditions described herein, in particular a metabolic disorder. The terms diseases, disorders and conditions are used interchangeably.

An IL18 antagonist, specifically an antigen binding protein, of the disclosure, may be used for treating or preventing a metabolic disorder. Use of an IL18 antagonist, specifically an antigen binding protein, of the disclosure in the manufacture of a medicament for preventing or treating a metabolic disorder is also provided.

A “metabolic disorder” is any disorder which is defined by an imbalance in metabolism of substances in the body including, but not limited to, carbohydrates, amino acids, organic acids, fatty acids, mitochondria, steroids. Metabolic disorders include the following non-limiting examples: insulin resistance, Type 2 diabetes mellitus (T2DM), obesity, metabolic syndrome, dislipidaemia, acute pancreatitis, liver failure, and co-morbidities associated with T2DM (e.g. atherosclerosis, cardiovascular diseases). In an embodiment, the metabolic disorder is T2DM.

In an embodiment an IL18 antagonist of the disclosure reduces glucose levels in a human patient.

IL-18 antagonists of the disclosure may improve peripheral insulin resistance, improve glycaemic control (i.e. maintenance of a target range for fasting blood glucose of below 8.9 mmol/L, in particular between 3.9-7.2 mmol/L), protect beta cells and prevent loss of beta cell function, improve pancreatic function (assessed by measuring the response of the pancreas to secretin), reduce body weight (via general metabolic improvement), improve cardiovascular health (assessed by measurement of plasma triglycerides, lidids, CRP, blood pressure and BMI), and/or slow disease progression without any of the foregoing causing hypoglycaemia (i.e. blood glucose falls below 3 mmol/L).

In an embodiment of the disclosure, an IL-18 induced increase in gene expression of any one or more or all of IL-6, STAT3, SOCS3, JAK2, MCP-1 (CCL2), and MCP-4 (CCL13) is reversed or partially reversed by an IL-18 antagonist of the disclosure, e.g. H1L2 (SEQ ID NO:7 and SEQ ID NO:11), in ex-vivo stimulated healthy volunteer blood.

In an embodiment of the disclosure, an IL-18 induced decrease in gene expression of IRS2, PPAR gamma, leptin and the leptin receptor is reversed or partially reversed by an IL-18 antagonist of the disclosure, e.g. H1L2 (SEQ ID NO:7 and SEQ ID NO:11), in ex-vivo stimulated healthy volunteer blood.

In an embodiment, the IL-18 antagonist of the disclosure does not reach the central nervous system (CNS) in appreciable quantities, but instead exerts its therapeutic effect by acting in the periphery.

The pharmaceutical preparation may comprise an IL-18 antagonist of the disclosure, e.g. an antigen binding protein, in combination with a pharmaceutically acceptable carrier. The IL-18 antagonist may be administered alone, or as part of a pharmaceutical composition.

Typically such compositions comprise a pharmaceutically acceptable carrier as known and called for by acceptable pharmaceutical practice, see e.g. Remingtons Pharmaceutical Sciences, 16th edition (1980) Mack Publishing Co. Examples of such carriers include sterilised carriers such as saline, Ringers solution or dextrose solution, optionally buffered with suitable buffers to a pH within a range of 5 to 8.

Pharmaceutical compositions may be administered by injection or continuous infusion (e.g. intravenous, intraperitoneal, intradermal, subcutaneous, intramuscular or intraportal). Such compositions are suitably free of visible particulate matter. In an embodiment, a pharmaceutical composition of the disclosure is administered via subcutaneous injection. In a further embodiment, a pharmaceutical composition of the disclosure is administered intradermally. Such intradermal administration may be achieved via injection with a single needle inserted at an angle of approximately 15° from the skin (Mantoux procedure), using patch technology (e.g. multitude of microneedles or abrasive surfaces) or other suitable means. When the IL-18 antagonist is a protein, the pharmaceutical composition may comprise between 0.01 mg to 10 g of protein, for example between 5 mg and 1 g of protein. Alternatively, the composition may comprise between 5 mg and 500 mg, for example between 5 mg and 50 mg.

Methods for the preparation of such pharmaceutical compositions are well known to those skilled in the art. Pharmaceutical compositions may comprise between 1 mg to 10 g of protein in unit dosage form, optionally together with instructions for use. Pharmaceutical compositions may be lyophilised (freeze dried) for reconstitution prior to administration according to methods well known or apparent to those skilled in the art. Where the IL-18 antagonist is an anti-IL-18 antibody and the antibody has an IgG1 isotype, a chelator of copper, such as citrate (e.g. sodium citrate) or EDTA or histidine, may be added to the pharmaceutical composition to reduce the degree of copper-mediated degradation of antibodies of this isotype, see EP0612251. Pharmaceutical compositions may also comprise a solubiliser such as arginine base, a detergent/anti-aggregation agent such as polysorbate 80, and an inert gas such as nitrogen to replace vial headspace oxygen.

Effective doses and treatment regimes for administering the IL-18 antagonist are generally determined empirically and may be dependent on factors such as the age, weight and health status of the patient and disease or disorder to be treated. Such factors are within the purview of the attending physician. Guidance in selecting appropriate doses may be found in e.g. Smith et al (1977) Antibodies in human diagnosis and therapy, Raven Press, New York.

The dosage of IL-18 antagonist administered to a subject is generally between 1 μg/kg to 150 mg/kg, between 0.1 mg/kg and 100 mg/kg, between 0.5 mg/kg and 50 mg/kg, between 1 and 25 mg/kg or between 1 and 10 mg/kg of the subject's body weight. For example, the dose may be 10 mg/kg, 30 mg/kg, or 60 mg/kg. In an embodiment, H1L2 (SEQ ID NO:7 and SEQ ID NO:11) is administered to a subject at a dosage of between 1 and 5 mg/kg. In another embodiment, H1L2 (SEQ ID NO:7 and SEQ ID NO:11) is administered to a subject at a dosage of about 3 mg/kg. The IL-18 antagonist may be administered parenterally, for example subcutaneously, intravenously or intramuscularly.

The administration of a dose may be by slow continuous infusion over a period of from 2 to 24 hours, such as from 2 to 12 hours, or from 2 to 6 hours.

The administration of a dose may be repeated one or more times as necessary, for example, three times daily, once every day, once every 2 days, once a week, once a fortnight, once a month, once every 3 months, once every 6 months, or once every 12 months. In a particular embodiment of the disclosure, the administration of a dose is once a month. In a further embodiment, the administration of a dose is once every 6 months. The IL-18 antagonists may be administered by maintenance therapy, for example once a week for a period of 6 months or more. The IL-18 antagonists may be administered by intermittent therapy, for example for a period of 3 to 6 months and then no dose for 3 to 6 months, followed by administration of IL-18 antagonist again for 3 to 6 months, and so on in a cycle.

The IL-18 antagonist may be administered to the subject in such a way as to target therapy to a particular site. For example, the IL-18 antagonist may be injected locally subcutaneously or intravenously.

The IL-18 antagonist may be used in combination with one or more other therapeutically active agents, including metformin, rosiglitazone, phentermine, topiramate, orlistat (Xenical™, Alli™), GLP-1 receptor agonists (e.g. exenatide (Byetta™), liraglutide (Victoza™), albiglutide (Syncria™)) and/or PYY receptor agonists for the treatment of the diseases described herein.

When the IL-18 antagonist, e.g. antigen binding protein, is used in combination with other therapeutically active agents, the individual components may be administered either together or separately, simultaneously, sequentially, concurrently or consecutively, in separate or combined pharmaceutical formulations, by any convenient route. If administered separately or sequentially, the IL-18 antagonist and the therapeutically active agent(s) can be administered in any order.

The combinations referred to above may be presented for use in the form of a single pharmaceutical formulation comprising a combination as defined above optionally together with a pharmaceutically acceptable carrier or excipient.

When combined in the same formulation it will be appreciated that the components must be stable and compatible with each other and the other components of the formulation and may be formulated for administration. When formulated separately they may be provided in any convenient formulation, for example in such a manner as known for antigen binding proteins in the art.

When in combination with a second therapeutic agent active against the same disease, the dose of each component may differ from that when the IL-18 antagonist is used alone. Appropriate doses will be readily appreciated by those skilled in the art.

The IL-18 antagonist and the therapeutically active agent(s) can act synergistically. In other words, administering the IL-18 antagonist and the therapeutically active agent(s) in combination has a greater effect on the disease, disorder, or condition described herein than the sum of the effect of each alone.

The terms “individual”, “subject” and “patient” are used herein interchangeably. The subject is typically a human. The subject may also be a mammal, such as a mouse, rat or primate (e.g. a marmoset or monkey). The subject can be a non-human animal. The IL-18 antagonists also have veterinary use. The subject to be treated may be a farm animal for example, a cow or bull, sheep, pig, ox, goat or horse or may be a domestic animal such as a dog or cat. The animal may be any age, or a mature adult animal.

Treatment can be therapeutic, prophylactic or preventative. The subject will be one who is in need thereof. Those in need of treatment may include individuals already suffering from a particular medical disease in addition to those who may develop the disease in the future.

Thus, the IL-18 antagonist described herein can be used for prophylactic or preventative treatment. In this case, the IL-18 antagonist described herein is administered to an individual in order to prevent or delay the onset of one or more aspects or symptoms of the disease. The subject can be asymptomatic. The subject may have a genetic predisposition to the disease. A prophylactically effective amount of the IL-18 antagonist is administered to such an individual. A prophylactically effective amount is an amount which prevents or delays the onset of one or more aspects or symptoms of a disease described herein.

The IL-18 antagonist described herein may also be used in methods of therapy. The term “therapy” encompasses alleviation, reduction, or prevention of at least one aspect or symptom of a disease. For example, the IL-18 antagonist described herein may be used to ameliorate or reduce one or more aspects or symptoms of a disease described herein.

The IL-18 antagonist described herein is used in an effective amount for therapeutic, prophylactic or preventative treatment. A therapeutically effective amount of the IL-18 antagonist described herein is an amount effective to ameliorate or reduce one or more aspects or symptoms of the disease. The IL-18 antagonist described herein may also be used to treat, prevent, or cure the disease described herein.

The IL-18 antagonist described herein can have a generally beneficial effect on the subject's health, for example it can increase the subject's expected longevity.

The IL-18 antagonist described herein need not affect a complete cure, or eradicate every symptom or manifestation of the disease to constitute a viable therapeutic treatment. As is recognised in the pertinent field, drugs employed as therapeutic agents may reduce the severity of a given disease state, but need not abolish every manifestation of the disease to be regarded as useful therapeutic agents. Similarly, a prophylactically administered treatment need not be completely effective in preventing the onset of a disease in order to constitute a viable prophylactic agent. Simply reducing the impact of a disease (for example, by reducing the number or severity of its symptoms, or by increasing the effectiveness of another treatment, or by producing another beneficial effect), or reducing the likelihood that the disease will occur (for example by delaying the onset of the disease) or worsen in a subject, is sufficient.

IL-18 antagonists described herein may be used in treating or preventing any metabolic disorder disclosed herein.

The term “therapeutically effective amount” refers to an amount (dose) of a substance, e.g. an IL-18 antagonist, that is sufficient to prevent, inhibit, halt, or allow an improvement in the disease being treated.

Accordingly, the disclosure provides methods of treating and/or preventing the above mentioned diseases comprising the step of administering a therapeutically effective amount of an IL-18 antagonist, e.g. an anti-IL18 antigen binding protein, to a patient in need thereof.

Within this specification the disclosure has been described, with reference to embodiments, in a way which enables a clear and concise specification to be written. It is intended and should be appreciated that embodiments may be variously combined or separated without parting from the disclosure.

All publications, including but not limited to patents and patent applications, cited in this specification are herein incorporated by reference as though fully set forth.

The disclosure is further described, for the purposes of illustration only, in the following examples.

### EXAMPLES

### Example 1

The blood from 10 healthy volunteer donors was split into 4 aliquots per donor and stimulated ex-vivo with either: A) Synagis™ 1 ug/ml (control anti-RSV IgG), B) IL-18 50 ng/ml, C) IL-18 50 ng/ml+H1L2 1 ug/ml or D) H1L2 1 ug/ml, in order to determine the effects of H1L2 (SEQ ID NO:7 and SEQ ID NO:11) on IL-18 induced gene expression. Samples for each treatment group were hybridised to Affymetrix™ U133_plus_{—}2.0 whole genome human microarrays. The following comparisons were performed:
Synagis™ IgG 1 ug/ml vs IL-18 50 ng/ml: to determine the effect of IL-18 on gene expression in blood
IL-18 50 ng/ml vs IL-18 50 ng/ml+H1L2 1 ug/ml: to determine the effects of H1L2 on IL-18 induced gene expression
Synagis™ IgG 1 ug/ml vs H1L2 1 ug/ml: to determine the effect of H1L2 on gene expression in blood in the absence of stimulus

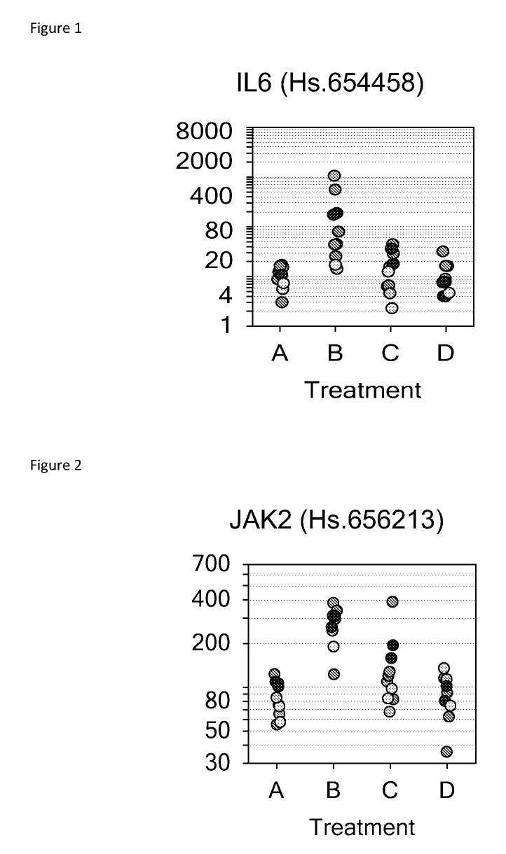

IL-18 stimulated ex-vivo blood showed a 9-fold (P<0.0001) increase in IL-6 expression, and the addition of H1L2 neutralised this effect (7 fold decrease when comparing IL-18+H1L2 data to IL-18 alone data). Data is shown in FIG. 1.

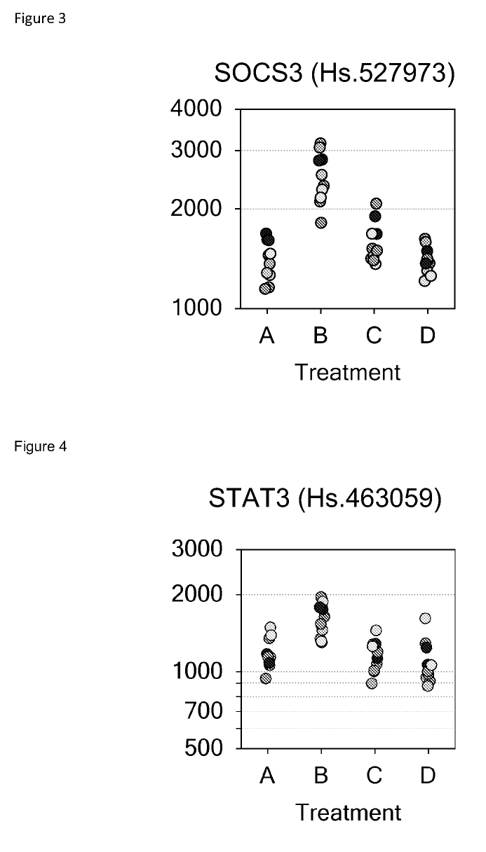

Furthermore, JAK2, SOCS3 and STAT3 showed 3.2 fold, 3.1 fold and 1.7 fold increases in expression with IL-18 stimulation respectively. The addition of H1L2 partially neutralised these effects: JAK2 with a 2.1 fold decrease, SOCS3 with a 1.9 fold decrease and STAT3 with a 1.6 fold decrease when comparing IL18+H1L2 to IL-18 alone. Data is shown in FIGS. 2, 3 and 4.

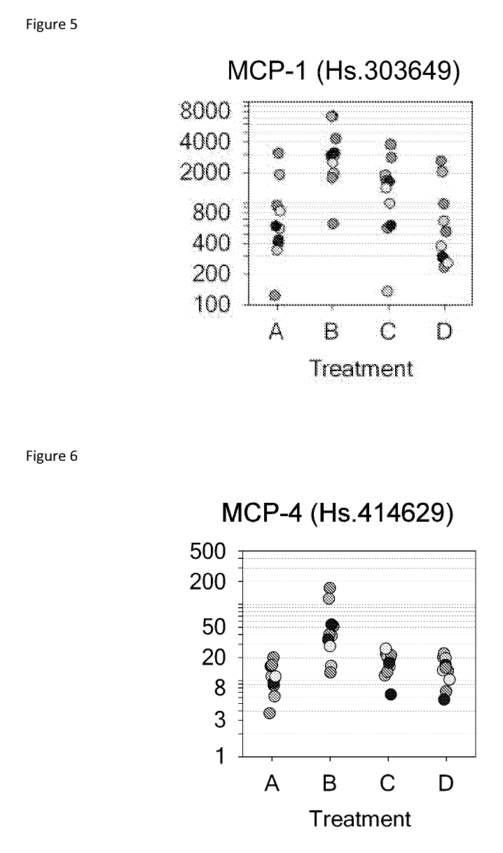

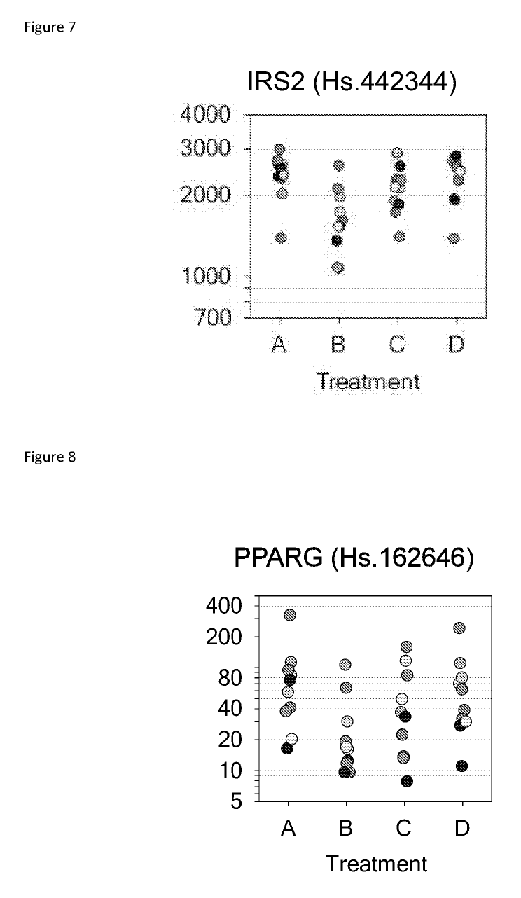

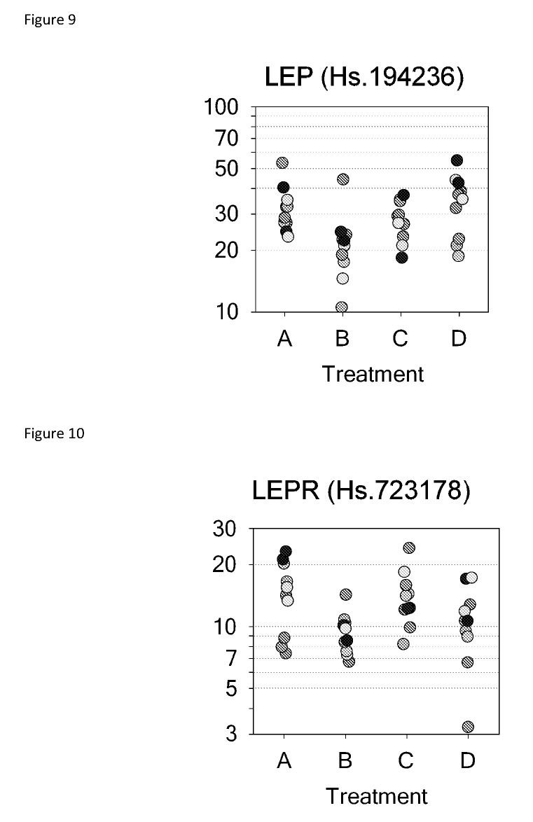

In addition, MCP-1, and MCP-4 showed a 4.4 and 4.1 fold increase in expression with IL18 stimulation respectively. These increases were partially reversed with the addition of H1L2, showing a 2.6 and 2.5 fold decrease when comparing IL18+H1L2 to IL18 alone (see FIG. 5-6). PPAR gamma and IRS2 showed a 2.9 and 1.5 fold decrease respectively with IL18 stimulation with effects being partially neutralised with the addition of H1L2 (see FIG. 7-8), showing 1.7 and 1.4 fold increase when comparing IL18+H1L2 against IL18 alone. Finally, leptin and the leptin receptor both showed a 1.5 fold decrease in expression following IL-18 stimulation. These effects were also neutralised by H1L2 (see FIG. 9-10).

This study was well powered with 90% confidence power to detect a 1.1 fold change in 90% of the probes on the microarray.

This data indicates that IL-18 induces expression of IL-6 and its key signalling molecules in blood and that these effects can be neutralised by H1L2.

### Example 2

The utility of infused or subcutaneously administered recombinant human IL-18 (rhIL-18) for the treatment of a range of cancers has been investigated in three Phase I studies and one Phase II study. Blood glucose and laboratory adverse event data from these studies were reviewed to explore any potential link between infusion of rhIL-18 and subsequent changes in glucose metabolism. In all phase I studies, patients with concomitant medical conditions such as diabetes were eligible for participation provided their disease was considered stable by the principle investigator and the patient had been receiving treatment for at least 6 months.

Supraphysiological levels of rhIL-18 were achieved in the plasma of patients over a 30-40 hour period at all dose levels investigated in these studies.

The most common clinical chemistry abnormality encountered during dosing of recombinant human IL-18 (rhIL-18) in these studies was hyperglycaemia.

Across monotherapy studies completed to date 38-68% of patients treated experienced at least Grade 1 (CTC criteria) hyperglycemia AE (>ULN-8.9 mmol/L) depending on protocol eligibility criteria (i.e. whether diabetic subjects were excluded or not).

Across the three completed and reported dose finding studies (n=72), 8 (7%) patients experienced a Grade 3 (>13.9-27.8 mmol/L) hyperglycaemic event and 1 patient experienced Grade 4 (>27.8 mmol/L) hyperglycemia. All 9 of these patients were diagnosed as diabetic at the time of randomisation.

Whilst there was no clear dose-relationship with plasma glucose levels, consistent timing of hypoglycaemia AEs relative to dosing (5-10 days post-infusion), in addition to the magnitude of increase in blood glucose, suggest these events were related to dosing with rhIL-18. These data indicate a link between IL-18 and elevated plasma glucose levels which in turn suggest that IL-18 antagonist may represent a useful treatment for metabolic disorders in which plasma glucose is elevated e.g. T2DM, obese T2DM and obesity.

### Example 3

An IL-18 antagonist of the disclosure may be used in the DIO (diet-induced obesity) mouse model at different doses to investigate its affect on weight loss as well as glucose levels, insulin levels and other metabolic parameters related to obesity and diabetes.

In this model C57bl/6 male mice reach a weight of c. 40-45 g when fed a 45% fat diet for 18-20 weeks. IL-18 antagonists are then dosed once or several times and the mice are weighed every day until the end of the study. Cardiac bleeds are collected from mice following terminal anesthesia and analysis of several markers is performed, including ALP, ALT, AST, GLDH, bilirubin, glucose, insulin, urea, creatinine, total protein, albumin, calcium, cholesterol, triglyceride, phosphate, sodium, potassium, chloride and ketones (both hydroxybutyrate and acetoacetate where possible). Tissues may be taken to assess histopathology for safety assessment and brain tissues for immunohistochemistry.

Given the results shown in examples 1 and 2 above, we expect the outcome of these murine studies to be a decrease in glucose and insulin levels in mice treated with an IL-18 antagonist, together with an impact on weight loss.

### Example 4

A first-time-in-human (FTIH) study (i.e. a single-blind, randomised, placebo-controlled study) to investigate the safety, tolerability, pharmacokinetics of single doses of intravenously infused H1L2 in healthy and obese subjects was carried out. Metabolic pharmacodynamics were also assessed in the obese subjects.

### Methodology

The study consisted of 2 parts. Part 1 consisted of 5 cohorts of healthy subjects (n=5-15 per cohort) and Part 2 consisted of 3 cohorts of obese subjects (n=5-12 per cohort); obese subjects being defined as those having a BMI of 30-40, but otherwise healthy. Each cohort participated in a single study session.

Both parts were conducted single-blind and with a placebo control. Within each cohort, allocation of subjects to placebo or active treatment was randomised.

The starting dose for Part 1 was 0.008 mg/kg and dose escalation proceeded to a maximum dose of 3.0 mg/kg. Dosing in Part 2 did not start until dosing to 1 mg/kg was completed for Part 1 and the preliminary safety and PK data had been reviewed.

To investigate the effect of H1L2 on cell mediated inflammation, a delayed type hyper-sensitivity (DTH) approach was included for the 1 mg/kg and 3 mg/kg cohorts of the healthy subjects in Part 1. Candin® is a Candida albicans skin test antigen that triggers a DTH inflammatory response when administered intradermally (Allermed Laboratories). 27 healthy volunteers who were confirmed DTH responders (>5 mm induration in response to a 0.1 mL Candin® injected intradermally into the volar surface of the arm), were recruited into the 1 mg/kg (H1L2 n=9, placebo n=3) and 3 mg/kg (H1L2 n=9, placebo n=6) healthy subject cohorts. Subjects enrolled into these cohorts received a second intradermal injection of Candin® on day 3 of the study (48 h after H1L2 dosing). In addition to induration and erythema assessments at 24 h and 48 h post Candin® challenge, 3 skin biopsies were taken from each subject. 2 mm or 3 mm punch biopsies were taken from an uninvolved region at screening, the centre of the DTH induration at screening (48 h post Candin® challenge), and the centre of the DTH induration on repeat challenge (48 h post Candin® challenge, 96 h post H1L2 dosing). RNA was extracted from the biopsies, labelled and hybridised to Affymetrix U133_plus_{—}2.0 whole genome human microarrays. The data was analysed to identify genes with expression changes in response to Candin® DTH challenge that were modulated by H1L2 administration. Inflammatory genes previously identified as having a role in metabolic disorders were assessed in this model.

3 cohorts of obese male volunteers were included in Part 2. The starting dose was 0.25 mg/kg and dose escalation proceeded to a maximum dose of 3.0 mg/kg; reflecting the highest three doses of Part 1.

Given the potential utility of H1L2 in patients with metabolic disease, this study also investigated the effects of H1L2 on metabolic pharmacodynamic endpoints in obese subjects. The healthy obese subjects included in this study had raised insulin levels indicating potential insulin resistance.

The oral glucose tolerance test (OGTT) was used to assess the potential metabolic effects of different doses of H1L2 in obese subjects. After ingestion of a 75 g oral glucose challenge, there is a rapid rise in insulin secretion (first phase response), which is followed by a more sustained release of the hormone (second phase). During this secretion process, C-peptide, or connecting peptide, is split from pro-insulin, the insulin precursor molecule, and is produced in equimolar amounts to insulin. In the bloodstream, C-peptide has a long half-life, because, unlike insulin, it is not subject to hepatic clearance. Blood samples were collected for 180 min so that the first and second phases of insulin secretion could be derived by modelling the C-peptide and insulin kinetics data during the OGTT. In addition, insulin sensitivity was calculated from the rate of appearance and disappearance of the ingested glucose.

### Results

DTH challenge skin resulted in a 113-fold (P<0.0001) increase in IL6 expression (123-fold increase P<0.0001 in 1 mg/kg cohort and 101-fold increase P<0.0001 in 3 mg/kg cohort). Treatment with H1L2 3 mg/kg prior to repeat challenge attenuated IL6 expression by 3.4-fold (P<0.0001) which is 1.8 times greater reduction (P=0.055) than placebo. This effect was not significant with treatment of H1L2 1 mg/kg (1.6 fold attenuation P=0.098).

Furthermore SOCS3 and STAT3 showed a significant (P<0.0001) 14-fold and 6-fold increase in expression with DTH respectively. SOCS3 DTH induced expression was attenuated by 1.9-fold with H1L2 3 mg/kg treatment, which was 1.4 times greater attenuation (P<0.05) than placebo. STAT3 DTH induced expression was attenuated by 1.3-fold (P<0.01) with H1L2 3 mg/kg, which was 1.4 times greater attenuation (P<0.01) than placebo. These effects were not observed with treatment of 1 mg/kg H1L2.

In addition, LEPR showed a 5-fold decrease (P<0.0001) in expression with DTH challenge. The decrease in LEPR due to DTH was attenuated by 1.5-fold (P<0.01) with H1L2 3 mg/kg. This was 1.4 times (P=0.084) greater attenuation than placebo.

Preliminary analysis showed that H1L2 also decreased glucose levels in the OGTT in obese subjects, these effects appeared more marked in subjects who had glucose levels above the upper level of normal suggesting that H1L2 may show larger effects in a more severe population such a patients with T2DM. There was also evidence that the insulin effects mirrored those observed on glucose levels in this sub-set of patients.

### Conclusions

These data indicate that H1L2 can attenuate the expression changes of IL6, STAT3, SOCS3 and LEPR in an in-vivo model of cell mediated inflammation. In addition, the data indicate that H1L2 modulates various metabolic parameters, including glucose and insulin levels. Accordingly, anti-IL18 antagonists, specifically the antibody H1L2, show promise is treating metabolic disorders.

1-24. (canceled)

25. A method of treating a metabolic disorder in a patient comprising the step of:
a) administering an effective amount of an IL-18 antagonist selected from the group consisting of IL-18 binding protein and an anti-IL-18 antigen binding protein, to a patient having a metabolic disorder selected from the group consisting of type 2 diabetes, obesity, insulin resistance, metabolic syndrome, dislipidaemia, acute pancreatitis and liver failure;
whereby the metabolic disorder in the patient is treated.

26. The method of claim 25, wherein the anti-IL-18 antigen binding protein comprises a CDRH1 amino acid sequence as shown in SEQ ID NO: 1, a CDRH2 amino acid sequence as shown in SEQ ID NO: 2, a CDRH3 amino acid sequence as shown in SEQ ID NO: 3; a CDRL1 amino acid sequence as shown in SEQ ID NO: 4, a CDRL2 amino acid sequence as shown in SEQ ID NO: 5, and a CDRL3 amino acid sequence as shown in SEQ ID NO: 6.

27. The method of claim 26, wherein the anti-IL-18 antigen binding protein is humanized

28. The method of claim 27, wherein the anti-IL-18 antigen binding protein is an IgG1 antibody.

29. The method of claim 28, wherein the anti-IL-18 antigen binding protein has a KD of about 10 nM or less.

30. The method of claim 28, wherein the patient is a human.

31. The method of claim 30, wherein the anti-IL-18 antigen binding protein is administered once a month.

32. The method of claim 31, wherein the anti-IL-18 antigen binding protein is administered subcutaneously.

33. The method of claim 31, wherein the anti-IL-18 antigen binding protein is administered intradermally.

34. The method of claim 30, wherein the anti-IL-18 antigen binding protein is administered once every six months.

35. The method of claim 34, wherein the anti-IL-18 antigen binding protein is administered subcutaneously.

36. The method of claim 34, wherein the anti-IL-18 antigen binding protein is administered intradermally.

37. The method of claim 26, wherein the anti-IL-18 antigen binding protein comprises an antibody having a heavy chain amino acid sequence at least 90% identical to an amino acid sequence as shown in SEQ ID NO: 7 and having a light chain amino acid sequence at least 90% identical to an amino acid sequence as shown in SEQ ID NO: 11.

38. The method of claim 37, wherein the anti-IL-18 antigen binding protein is humanized

39. The method of claim 38, wherein the anti-IL-18 antigen binding protein is an IgG1 antibody.

40. The method of claim 39, wherein the anti-IL-18 antigen binding protein has a KD of about 10 nM or less.

41. The method of claim 39, wherein the patient is a human.

42. The method of claim 41, wherein the anti-IL-18 antigen binding protein is administered once a month.

43. The method of claim 42, wherein the anti-IL-18 antigen binding protein is administered subcutaneously.

44. The method of claim 42, wherein the anti-IL-18 antigen binding protein is administered intradermally.

45. The method of claim 41, wherein the anti-IL-18 antigen binding protein is administered once every six months.

46. The method of claim 45, wherein the anti-IL-18 antigen binding protein is administered subcutaneously.

47. The method of claim 45, wherein the anti-IL-18 antigen binding protein is administered intradermally.

48. The method of claim 37, wherein the anti-IL-18 antigen binding protein comprises an antibody having a heavy chain amino acid sequence as shown in SEQ ID NO: 7 and having a light chain amino acid sequence as shown in SEQ ID NO: 11.

49. The method of claim 48, wherein the anti-IL-18 antigen binding protein is humanized

50. The method of claim 49, wherein the anti-IL-18 antigen binding protein is an IgG1 antibody.

51. The method of claim 50, wherein the anti-IL-18 antigen binding protein has a KD of about 10 nM or less.

52. The method of claim 50, wherein the patient is a human.

53. The method of claim 52, wherein the anti-IL-18 antigen binding protein is administered once a month.

54. The method of claim 53, wherein the anti-IL-18 antigen binding protein is administered subcutaneously.

55. The method of claim 53, wherein the anti-IL-18 antigen binding protein is administered intradermally.

56. The method of claim 52, wherein the anti-IL-18 antigen binding protein is administered once every six months.

57. The method of claim 56, wherein the anti-IL-18 antigen binding protein is administered subcutaneously.

58. The method of claim 56, wherein the anti-IL-18 antigen binding protein is administered intradermally.


SEQUENCE LISTING (13 sequences)

SEQ ID NO: 1 - protein, 5 aa, Artificial Sequence
  CDRH1 Amino acid sequence identified using molecular biology techniques.
GYYFH

SEQ ID NO: 2 - protein, 17 aa, Artificial Sequence
  CDRH2 Amino acid sequence identified using molecular biology techniques.
RIDPEDDSTKYAERFKD

SEQ ID NO: 3 - protein, 17 aa, Artificial Sequence
  CDRH3 Amino acid sequence identified using molecular biology techniques.
WRIYRDSSGRPFYVMDA

SEQ ID NO: 4 - protein, 11 aa, Artificial Sequence
  CDRL1 Amino acid sequence identified using molecular biology techniques.
LASEDIYTYLT

SEQ ID NO: 5 - protein, 7 aa, Artificial Sequence
  CDRL2 Amino acid sequence identified using molecular biology techniques.
GANKLQD

SEQ ID NO: 6 - protein, 9 aa, Artificial Sequence
  CDRL3 Amino acid sequence identified using molecular biology techniques.
LQGSKFPLT

SEQ ID NO: 7 - protein, 456 aa, Artificial Sequence
  H1 Amino acid sequence identified using molecular biology techniques.
QVQLVQSGAEVKKPGASVKVSCKVSGEISTGYYFHWVRQAPGKGLEWMGRIDPEDDSTKYAERFKDRVTMTEDTSTDTAYMELSSLRSEDTAVYYCTTWRIYRDSSGRPFYVMDAWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 456 aa, Artificial Sequence
  H2 Amino acid sequence identified using molecular biology techniques.
QVQLVQSGAEVKKPGASVKVSCKVSGEISTGYYFHWVRRRPGKGLEWMGRIDPEDDSTKYAERFKDRVTMTEDTSTDTAYMELSSLRSEDTAVYYCTTWRIYRDSSGRPFYVMDAWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 456 aa, Artificial Sequence
  H3 Amino acid sequence identified using molecular biology techniques.
QVQLVQSGAEVKKPGASVKVSCKVSGEISTGYYFHFVRRRPGKGLEWMGRIDPEDDSTKYAERFKDRVTMTADTSTDTAYMELSSLRSEDTATYFCTTWRIYRDSSGRPFYVMDAWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 214 aa, Artificial Sequence
  L1 Amino acid sequence identified using molecular biology techniques.
DIQMTQSPSSVSASVGDRVTITCLASEDIYTYLTWYQQKPGKAPKLLIYGANKLQDGVPSRFSGSGSGTDFTLTISSLQPEDFATYYCLQGSKFPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 214 aa, Artificial Sequence
  L2 Amino acid sequence identified using molecular biology techniques.
DIQMTQSPSSVSASVGDRVTITCLASEDIYTYLTWYQQKPGKAPKLLIYGANKLQDGVPSRFSGSGSGTDYTLTISSLQPEDFATYYCLQGSKFPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 214 aa, Artificial Sequence
  L3 Amino acid sequence identified using molecular biology techniques.
DIQMTQSPSSVSASVGDRVTITCLASEDIYTYLTWYQQKPGKAPQLLIYGANKLQDGVPSRFSGSGSGTDYTLTISSLQPEDEGDYYCLQGSKFPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 193 aa, Artificial Sequence
  IL-18
MAAEPVEDNCINFVAMKFIDNTLYFIAEDDENLESDYFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED